(19) United States
(12) Patent Application Publication
(10) Pub. No.: US 2008/0132876 A1
(43) Pub. Date: Jun. 5, 2008

(54) Connector System for Sterile Connection

(75) Inventor: Thomas J. FELT, Boulder, CO (US)

Correspondence Address: GAMBRO BCT, INC;IP DEPARTMENT, 10810 WEST COLLINS AVE, LAKEWOOD, CO, 80215, US

(73) Assignee: GAMBRO BCT, INC., Lakewood, CO (US)

(21) Appl. No.: 11/835,577

(22) Filed: Aug. 8, 2007

Related U.S. Application Data
(60) Provisional application No. 60/868,643, filed on Dec. 5, 2006.

(51) Int. Cl. A61M 39/04; A61M 39/00

(52) U.S. Cl. 604/411; 285/3

(57) ABSTRACT

This invention is directed toward a connector system for the fluid connection of sterile systems comprising a male luer connecting element and a female luer connecting element. Both the male and female luer connecting elements have openable ends aseptically sealed closed by piercable membranes.

### CROSS REFERENCE TO RELATED APPLICATIONS

This application claims the benefit of U.S. Provisional Application No.: 60/868,643 filed Dec. 5, 2006.

### FIELD OF THE INVENTION

This invention relates to the field of sterile connectors for tubing, including medical tubing.

### BACKGROUND

In the medical field it is often necessary to connect medically associated tubing and other accessories together in a sterile manner. An important area of application for connectors is connecting containers containing various fluids to other containers, or connecting the ends of two portions of tubing to each other for the purpose of providing extension lines.

In the field of blood banking for example, it is desirable to connect containers of blood or blood components to containers containing solutions for the storage of the blood or blood components. Generally, some type of previously sterilized disposable is provided. This disposable can include bags, including collection and transfer bags as well as connecting tubing. However, it may be desirable after the initial sterilization of the bags and tubing sets and/or during use, to attach additional bags, filters, additional lengths of tubing or sample containers. In such a situation, it is necessary to provide a sterile connection to prevent contaminating any collected blood product or the disposable set itself.

One known method of making a sterile connection is through the use of a piercable membrane. Such a membrane acts as a physical barrier, retaining the sterility of its associated tubing until pierced upon connection.

One type of membrane connector described in U.S. Pat. No. 6,234,538 includes two piercable membranes with the first membrane being on one tubing line and the second membrane being on a connector element.

A need still exists however for a simple improved sterile connector which may be pre-connected to tubing so that additional elements may be added after a sterilization procedure.

### SUMMARY OF THE INVENTION

This invention is directed toward a connector system for the fluid connection of sterile systems. The connector system includes a male luer connecting element adapted for connection to a female luer connecting element. The male luer connecting element includes a male luer taper and a rigid external housing column. The rigid external housing column circumferentially surrounds the male luer taper and has an openable end which has a first piercable membrane aseptically sealing the openable end. The female luer connecting element includes a female luer taper, a collapsible column which circumferentially surrounds the female luer taper and has an openable end, and a rigid external collar which circumferentially surrounds the collapsible column and has an open end. A second piercable membrane asceptically seals the female luer taper openable end.

### BRIEF DESCRIPTION OF THE FIGURES

FIG. 1a shows a cross sectional view of the disconnected male connector of this invention.

FIG. 1b shows a cross sectional view of the disconnected female connector of this invention.

FIG. 2 shows a cross sectional view of the male and female connectors in an initial position before connection.

FIG. 3 is a cross sectional view showing the male and female connectors in a connected position.

FIG. 4 is a tubing set which may be used with the present invention.

### DETAILED DESCRIPTION

This invention is generally related to a simple connector which may be incorporated into a pre-sterilized disposable set such as those used for blood collection and/or separation.

Although the instant application is described with respect to connecting tubing which may be used in blood banking applications, it is understood that the current invention can also be used to connect tubing to medical equipment such as dialyzers, dialysis machines, apheresis machines, catheters, cannulas, peritoneal bags and other medical as well as non-medical applications for which maintaining sterility in tubing lines is desirable.

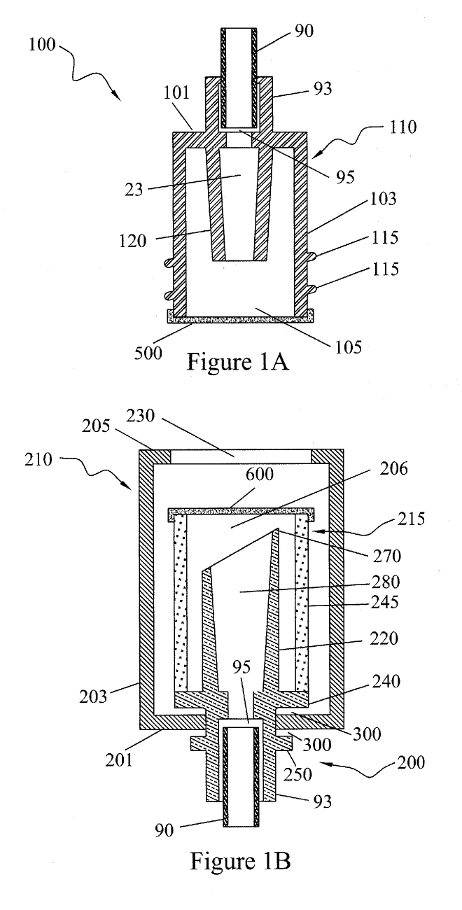

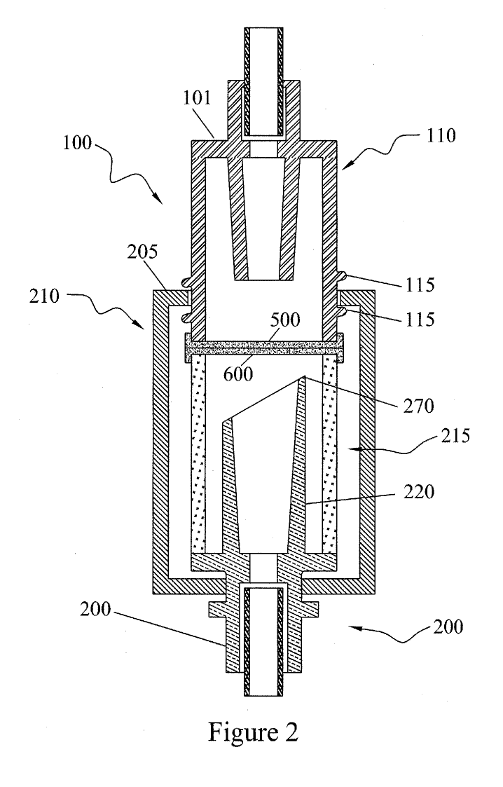

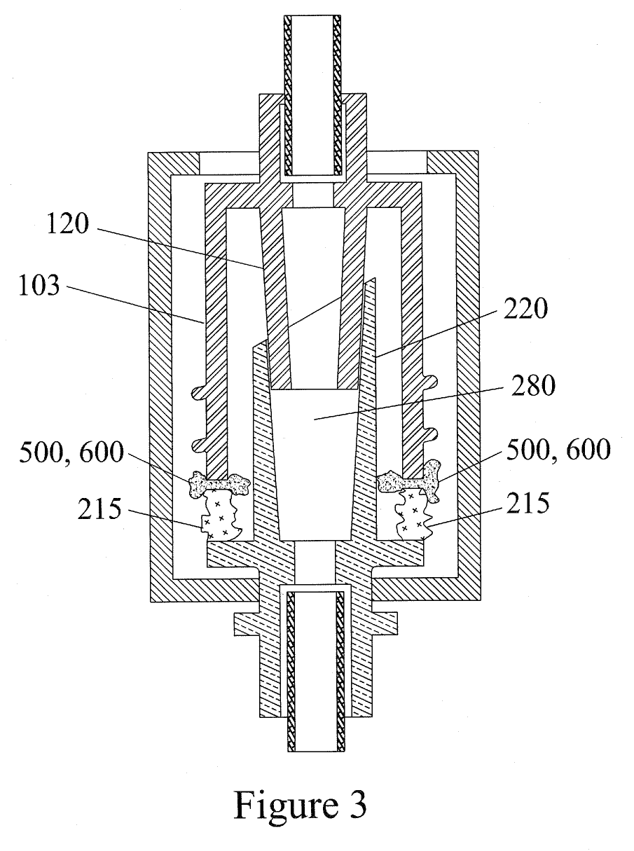

FIGS. 1a and 1b show two connector ends 100, 200 respectively, which may be connected together as described below (see FIGS. 2 and 3) to form a sterile connection. Element 100 is a male connector and element 200 is the corresponding female connector. It should be noted that like numerals denote like elements.

### Male Connector

Male connector element 100 includes a rigid external housing column 110, which includes a base portion 101 and a generally cylindrical side portion 103. External housing column 110 has an openable end 105 which is distal to the base portion 101. External housing column 110 circumferentially encloses a male luer taper 120. The male luer taper 120 extends from the base portion 101 outwardly towards the openable end 105 of the housing.

The base portion 101 also includes a generally cylindrical socket connector 93 which has an opening 95. The generally cylindrical socket connector 93 and opening 95 extend co-axially from base portion 101 in an opposite direction than the male luer taper 120.

Male luer taper 120 encloses passageway 23. Passageway 23 is open at both ends of the male luer taper to fluidly connect to opening 95 in generally cylindrical socket connector 93.

Tubing 90 is attached to connector 100 inside the opening 95 of generally cylindrical socket 93. Once tubing 90 is inserted inside opening 95 of generally cylindrical socket 93, tubing 90 and passageway 23 of male luer taper 120 line up to create a fluid flow passageway.

The tubing 90 is affixed to the connector 100 by solvent bonding or other known methods. This is generally done prior to sterilization of the tubing or conduit 90. Tubing 90 may be part of a disposable set having additional components. Once the connector and tubing are connected, a flowing medium, such as blood, can flow from tubing 90 through male luer connector 100.

The generally cylindrical side portion 103 of the rigid housing includes external threads 115. External threads 115 are located towards the openable end of the generally cylindrical side 103. The threads 1 15 engage with the lip 205 of the rotating guide/drive collar 203 (see FIG. 1b as described below) of the female luer connector 200 during the connection process.

Openable end 105 of connector element 100 may be aseptically sealed closed by an adhesive coated piercable membrane 500. Piercable membrane 500 prevents unwanted penetration of external contaminants into the sterile interior of the connector.

The piercable membrane 500 may also have a disinfecting quality on any contaminants which become stuck in the adhesive coating. The piercable membrane may be made of any material which may be made of or coated with adhesive and easily pierced.

The piercable membrane 500 may also be coated with a gel which traps any unwanted contaminants.

The male connector 100 may be made of plastic, e.g. polycarbonate, acrylonitrile butadiene styroles, polyethylenes, polypropylenes, polystyroles, polymethyl methacrylates, polysulfones, methyl methacrylate butadiene styroles, methyl methacrylate acrylonitrile butadiene styroles or any other plastic material which is compatible with the tubing material coming into contact with the connector 100, with the adhesive (if used) or solvent bonding, as well as with the fluid flowing therethrough.

### Female Connector

As shown in FIG. 1b, female connector element 200 also has a rigid external housing column or collar 210 which includes a base portion 201 and a generally cylindrical side portion 203. External rigid housing 210 has an open end 230. The generally cylindrical side portion 203 has walls which extend in a direction away from the base portion 201 and end in a lip 205. Lip 205 extends into a portion of the open end 230. Lip 205 engages with the external threads 115 of housing 110 of the male luer connector 100 as discussed above.

Connector 200 also includes a generally cylindrical socket connector 93 which has an opening 95. The generally cylindrical socket connector 93 and opening 95 extends in an opposite direction from the female luer taper 220. Generally cylindrical socket portion 93 has a lip 250 which forms one side of a groove 300 that the base portion 201 of rotating collar 210 rotates in.

The rotating guide/drive collar 210 circumferentially surrounds a generally cylindrical collapsable column 215 or cover for female luer connector 220. Generally cylindrical collapsible column 215 has a rigid base portion 240 and a generally cylindrical collapsible side portion 245. The generally cylindrical collapsible side portion 245 is made of a flexible material such as latex which is easily collapsible upon application of a force.

Generally cylindrical collapsible column 215 has an openable end 206 which is sealed with an adhesive coated piercable membrane 600. Piercable membrane 600 prevents unwanted entry of contaminants into the sterile interior of the collapsible column and female luer tip.

The permeable membrane 600 may also have a disinfecting effect on any contaminants which become stuck in the adhesive coating. The piercable membrane may be made of any material which may be made of or coated with adhesive and easily pierced.

The piercable membrane 500 may also be coated with a gel which traps any unwanted contaminants.

Rigid base portion 240 of collapsible column 215 forms the other side of the groove 300 that rotating guide/drive collar 210 rotates around.

The collapsible column 215 surrounds a rigid female luer taper 220 having a spike tip 270. The female luer taper 220 extends from the rigid base portion 240 of the circular collapsible column 215 outwardly towards openable end 206 of the collapsible column 215.

Female luer taper 220 surrounds passageway 280. Passageway 280 is open at both ends to fluidly connect to opening 95 in circular socket connection 93.

Connector 200 is attached to tubing or conduit 90 via the opening 95 in the circular socket 93. The tubing 90 is affixed to the connector 200 by solvent bonding or other known methods. This is generally done prior to sterilization of tubing or conduit 90. Tubing 90 may be part of a disposable set having additional components. Once the connector and tubing are connected, a flowing medium such as blood, can flow from tubing 90 through female luer connector 200.

The female connector 200 may be made of plastic, e.g. polycarbonate, acrylonitrile butadiene styroles, polyethylenes, polypropylenes, polystyroles, polymethyl methacrylates, polysulfones, methyl methacrylate butadiene styroles, methyl methacrylate acrylonitrile butadiene styroles or any other plastic material which is compatible with the tubing material 90 coming into contact with the connector 200, with the adhesive (if used), as well as with the fluid flowing therethrough.

The connectors 100, 200 and attached tubing 90 may be sterilized using any means known in the art.

### Connection

Connector elements 100 and 200 are joined starting from the positions illustrated in FIG. 1.

The rotating guide/drive collar 210 of female luer connector 200 helps to guide the two adhesive faces together. As shown in FIG. 2, the membrane faces 500 and 600 are pressed together, trapping any contaminants which may be present into the adhesive gel. The membrane faces will also help prevent any additional non-sterile substances from entering between the luers.

As the connectors are pressed together, the membrane gels extrude out between the rigid housings 110, 210, forming a seal between the connectors. Once the membrane faces are joined, they will capture all the non-sterile substances between them, and prevent additional non-sterile substances from entering into the sterile portion of the connectors, compromising sterility.

The lip 205 on the end of the drive collar 210 screws into the external threads 115 on the male luer housing 110 to help push the spike tip 270 of the female luer 220 through the pressed together membranes 500, 600.

As shown in FIG. 3, as the two connectors 100, 200 are pushed together, the collapsable column 215 surrounding the female luer 220 collapses against the rigid base portion 240 without breaking into pieces. This is an important feature so no broken off pieces could come in contact with the interior of the fluid flow passage.

The membranes 500, 600 are pierced through by spike tip 270 and are pushed out of the way by rigid column 103 of the male connector 100. The rigid column 103 around the male luer 120 pushes the pierced-through membranes 500, 600 against the collapsed collapsible column 215. This design makes sure that the pierced-through membranes do not interfere with the connection of the two luers.

Once the membranes are pierced by the female luer spike tip 270, the male luer connector 120 fits snugly into passageway 280 of the female luer connector 220 forming a fluid connection, while maintaining sterility of the connectors.

The connectors according to the present invention guarantee a reliable sterile connection before, during and after the connection of the two connectors. The connectors may be used as part of a blood tubing set if it is desired to sterilely connect additional bags to the set.

### Disposable

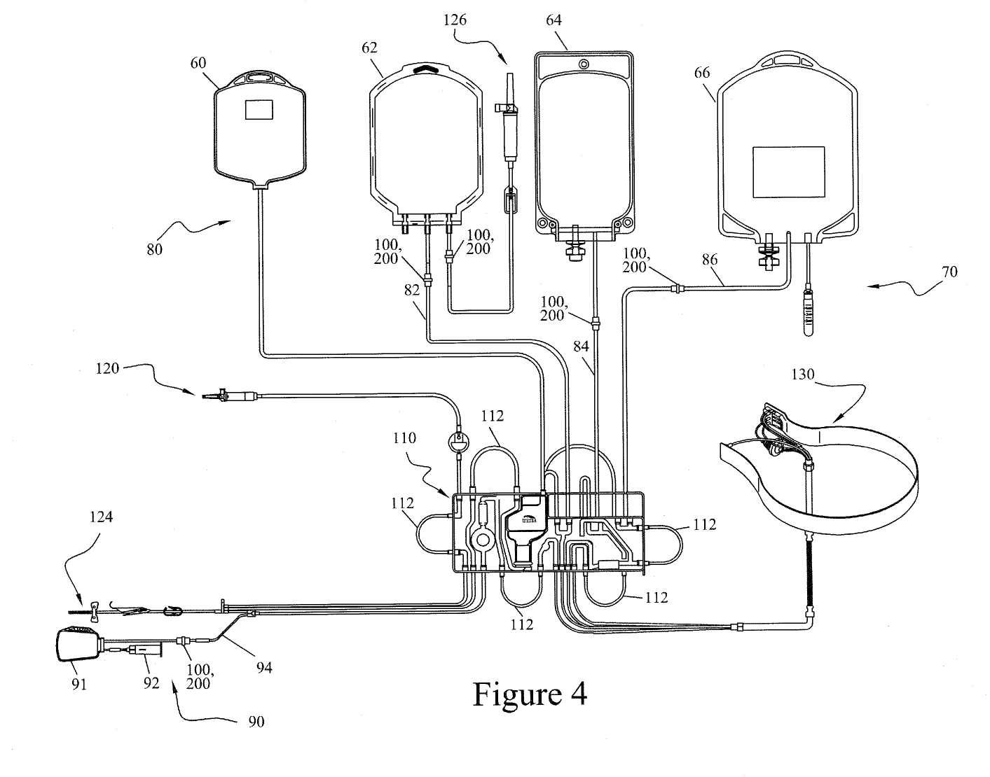

FIG. 4 shows a typical disposable set 70 for use on an apheresis machine. The disposable or tubing set 70 includes a blood processing vessel 130 for separating blood into various blood components. The set 70 includes a fluid flow cassette 110 having peristaltic pump loops 112, a receive and return line 124 for receiving and returning blood or blood components to or from a donor, an anticoagulant line 120 for adding anticoagulant to blood withdrawn from the donor, and a gas line 80 for receiving into bag 60 gas purged from the system.

A sample set 90 may also be part of the disposable set 70. Sample set 90 may be connected to the disposable set 70 using connectors 100 and 200. The sample set 90 includes line or conduit 94 through which a blood sample fluidly flows to sample bag 91. A blood sample may then be retrieved through sample device 92.

Line or conduit 82 leads to red blood cell bag 62. Connectors 100 and 200, such as the ones described above, may be used to connect a container containing storage solution (not shown) to red blood cell bag 62. Connectors 100, 200 may also be used to connect an additional bag to container 62 or to line 82 if additional red blood cells are collected, or to a leukoreduction filter if it is desired to leukoreduce the collected red blood cells.

Connectors 100, 200 may also be used to connect one or more plasma bags 64 to plasma line 84 and/or platelet bags 66 to platelet line 86.

It is understood that the connectors of this invention may be used on any tubing sets as well as for other uses involving medical or collapsible tubing as needed. It is further understood that such connectors may be useful on a variety of apheresis tubing as well as whole blood collection tubing. The connectors of the instant invention are appropriate to connect any tubing where a secure and sterile connection is needed.

It will be apparent to those skilled in the art that various modifications and variations can be made in the structure and methodology of the present invention, without departing from the spirit and scope of the invention. Thus, it is intended that the present invention cover the modifications and variations of this invention provided they come within the scope of the claims and their equivalents.

1. A connector system for the connection of sterile systems comprising:
a male luer connecting element adapted for connection to a female luer connecting element;
the male luer connecting element comprising:
a male luer taper;
a rigid external housing column;
the rigid external housing column circumferentially surrounds the male luer taper having an openable end; and
a first piercable membrane aseptically sealing the male luer taper openable end;
the female luer connecting element comprising;
a female luer taper;
a collapsible column which circumferentially surrounds the female luer taper and having an openable end;
a second piercable membrane asceptically sealing the female luer taper openable end; and
a rigid external collar which circumferentially surrounds the collapsible column and has an open end.

2. The connector system of claim 1 wherein the rigid external housing column of the male luer connecting element further comprises;
a base portion; and
a generally cylindrical side portion.

3. The connector system of claim 2 wherein the base portion of the rigid external housing column of the male luer connecting element farther includes a generally cylindrical socket connector having an opening extending co-axially from the base portion in an opposite direction from the male luer taper.

4. The connector system of claim 3 wherein the male luer taper of the male luer connecting element has a passageway fluidly connected to the opening in the generally cylindrical socket connector.

5. The connector system of claim 3 wherein the male luer connecting element further comprises a tubing extending from inside the opening of the generally cylindrical socket connector.

6. The connector system of claim 2 wherein the generally cylindrical side portion of the rigid external housing of the male luer connecting element further includes external threads.

7. The connector system of claim 1 wherein the first piercable membrane of the male luer connecting element is adhesive.

8. The connector system of claim 1 wherein the first piercable membrane of the male luer connecting element is coated with a gel.

9. The connector system of claim 1 wherein the rigid external collar of the female luer connecting element is capable of rotating.

10. The connector system of claim 1 wherein the rigid external collar of the female luer connecting element further comprises a base portion; and
a generally cylindrical side portion which extends in a direction away from the base portion and ends in a lip.

11. The connector system of claim 10 wherein the lip of the generally cylindrical side portion of the female luer connecting element is adapted to engage with the external threads of the generally cylindrical side portion of the rigid external housing column of the male luer connector element.

12. The connector system of claim 10 wherein the female luer connecting element further comprises a generally cylindrical socket connector having an opening extending co-axially from the base portion in an opposite direction from female luer taper.

13. The connector system of claim 12 wherein the generally cylindrical socket connector of female luer connecting element further comprises a lip which forms one side of a groove that the base portion of the rigid rotating collar is adapted to rotate in.

14. The connector system of claim 1 wherein the collapsible column of female luer connecting element further comprises a rigid base portion and a generally collapsible side portion.

15. The connector system of claim 14 wherein the female luer taper of female luer connecting element extends from the rigid base portion outwardly toward the openable end of the collapsible column.

16. The connector system of claim 1 wherein the female luer taper of female luer connecting element has a spike tip.

17. The connector system of claim 12 further comprising a tubing extending inside the opening of the generally cylindrical socket of female luer connecting element.

18. The connector system of claim 1 wherein the second piercable membrane of female luer connecting element is adhesive.

19. The connecting element of claim 1 wherein the second piercable membrane of female luer connecting element is coated with a gel.

20. A disposable for a blood processing apparatus comprising:
a blood processing vessel; and
at least one additional container
wherein the at least one additional container is connected to the blood processing vessel via a connector system for the connection of sterile systems comprising:
a male luer connecting element adapted for connection to a female luer connecting element;
the male luer connecting element comprising:
a male luer taper;
a rigid external housing column;
the rigid external housing column circumferentially surrounds the male luer taper having an openable end; and
a first piercable membrane aseptically sealing the male luer taper openable end;
the female luer connecting element comprising;
a female luer taper;
a collapsible column which circumferentially surrounds the female luer taper and having an openable end;
a second piercable membrane asceptically sealing the female luer taper openable end; and
a rigid external collar which circumferentially surrounds the collapsible column and has an open end.

21. The disposable of claim 20 wherein the at least one additional container is a blood product container.

22. The disposable of claim 20 wherein the at least one additional container is a container containing storage solution.

23. The disposable of claim 22 wherein the container containing storage solution is connected to a blood product container via the connector system of claim 20.

24. A method for fluidly connecting a first fluid line attached to a male luer connecting element having an adhesive face to a second fluid line attached to a female luer connecting element having an adhesive face comprising the steps of:
guiding the adhesive face of the male luer connecting element to the adhesive face of the female luer connecting element;
adhering the adhesive faces together to create adhered faces;
screwing the connectors together to push a spike tip of the female luer connecting element through the adhered faces to pierce the adhered faces;
connecting the male and female luers to fluidly connect the first fluid line to the second fluid line
wherein the screwing step further comprises screwing the connectors together to collapse a collapsible column surrounding the female luer without breaking the collapsible column into pieces.

25. The method of claim 24 wherein the adhering step further comprises the step of pressing the adhesive faces together to trap any non-sterile substances which may be present between the faces.

26. The method of claim 24 wherein the screwing step further includes the step of pushing the pierced through adhered faces of the luers against the collapsed collapsible column.
